(19) United States
(12) Patent Application Publication
(10) Pub. No.: US 2025/0093322 A1
(43) Pub. Date: Mar. 20, 2025

(54) CONE PENETROMETER TEST (CPT) TUBE

(71) Applicant: GO-Beheer B.V., Schiedam (NL)

(72) Inventors: Coen Johan Schoon, Tolbert (NL); Gerrit Tjerk De Vries, Schiedam (NL); Regina Usbeck, Bremerhaven (DE); Johannes Kässbohrer, Bremerhaven (DE)

(21) Appl. No.: 18/960,914

(22) Filed: Nov. 26, 2024

Related U.S. Application Data
(63) Continuation of application No. PCT/NL2023/050290, filed on May 23, 2023.

(30) Foreign Application Priority Data
Jun. 1, 2022 (NL) 2032052

(51) Int. Cl. G01N 33/24; G01N 25/00

(52) U.S. Cl. CPC G01N 33/24; G01N 25/00

(57) ABSTRACT

A cone penetrometer test (CPT) tube with a forward pointing cone equipped to be pushed into a soil at a controlled rate. At a fixed distance outside and adjacent to the tube, a longitudinally extending measurement probe is provided which is equipped for measuring thermal properties of the soil in which the tube is pushed. The longitudinally extending measurement probe extends along more than about 90% of the tube.

### CROSS-REFERENCE TO RELATED APPLICATIONS

This application is a continuation of International Patent Application No. PCT/NL2023/050290, filed May 23, 2023, entitled “A Cone Penetrometer Test (CPT) Tube”, which claims priority to and the benefit of Netherlands Patent Application No. 2032052, entitled “A Cone Penetrometer Test (CPT) Tube”, filed Jun. 1, 2022, and the specifications and claims thereof are incorporated herein by reference.

### BACKGROUND OF THE INVENTION

### Field of the Invention

The invention relates to a cone penetrometer test (CPT) tube with a forward pointing cone equipped to be pushed into a soil at a controlled rate.

### Background

Wikipedia mentions that such a cone penetrometer test (CPT) tube is developed in the fifties of the previous century at the Dutch Laboratory for Soil Mechanics in Delft to investigate soft soils. Based on this history it has also been called the “Dutch cone test”. Today, the CPT is one of the most used and accepted tools for soil investigation worldwide.

There is a need to complete the measurements that are possible with a cone penetrometer test (CPT) tube, with other measurements, in particular thermal measurements at relatively low depth of 0.3 m up to 10 m. Thermal measurements in connection with geology measurements are in general useful in oil and gas exploration, the offshore wind energy market, and in connection with gas hydrates, but such measurements are usually conducted at relatively larger depths and at great sea depth. In the known systems that are applied for those purposes, sensor-strings with a plurality of temperature sensors are vibrated into the ground.

### BRIEF SUMMARY OF THE INVENTION

It is an object of the invention to provide a method or system which is suitable for executing both a conventional cone penetrometer test measurement of a soil as well as a measurement of the thermal properties or conductivity of such soil. This is in particular desirable for assessment of a soil's suitability for cable burial onshore or in coastal zones with a sandy or clay sediment. The latter option in particular applies to offshore electric power cable burial and pipeline burial.

WO 2018/074928 discloses a probe arranged for subsurface penetration of a soil for measuring properties of the soil. The probe comprises a probe body and at least one fiber optical sensor. The at least one fiber optical sensor is arranged for measuring at least one of a cone resistance, sleeve friction, pore water pressure or inclination.

According to the invention a cone penetrometer test (CPT) tube is proposed with features according to one or more of the appended claims.

The cone penetrometer test (CPT) tube of the present invention comprises the feature that at a fixed distance outside and adjacent to the tube a longitudinally extending measurement probe is provided which is equipped for measuring thermal properties of the soil in which the tube is pushed, wherein the longitudinally extending measurement probe is fixed to the tube with two or more outriggers so as to ensure that the measurement probe can be pushed into the soil together with the tube while the tube and the probe maintain their relative position with reference to each other, and that the longitudinally extending measurement probe is provided with a heat supply portion and/or a cooling portion.

The heat supply portion and the cooling portion can be either passive or active. In the passive mode friction between the tube and probe and the soil can be used for heating. This can also be done active with dedicated heating elements. Active cooling is of course also possible.

Preferably the longitudinally extending measurement probe extends along at least 1 meter of the tube. With such a measurement instrument it is possible to simultaneously measure different parameters of the soil to be investigated.

Notable advantages of the combined tube and probe of the invention are the relatively quick mobilization and demobilization, the fact that deployment from small vessels is possible, and that the instrument requires minimal operator involvement enabling rapid and accurate testing. Further sediment disturbance is minimal which also contributes to accurate thermal property measurements. Because of the combined testing of the thermal properties with the conventional cone penetrometer test, measurement time is reduced by a factor two or more.

It is preferred that the tube and probe are mounted in a driving frame which is further provided with at least one centralizer and guide block for the tube and probe so as to be able to push the tube and probe in a controlled fashion into the soil. The driving frame is for instance provided with a set of driving rolls for driving the tube and probe, although other feasible driving options exist as well, such as driving chains driven by hydraulic engines. Another possible driving mode is for instance a conventional set of CPT rams as used onshore.

The invention will hereinafter be further elucidated with reference to the drawing of an exemplary embodiment of a combined cone penetrometer test (CPT) tube and thermal measurement probe according to the invention that is not limiting as to the appended claims. Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 is an illustration showing a side view of an instrument according to an embodiment of the present invention;

FIGS. 2A and 2B are illustrations showing two different side views of an instrument according to an embodiment of the present invention prior to entering into a soil to be investigated;

FIGS. 3A and 3B are illustrations showing two different side views of the instrument according to an embodiment of the present invention after entering into the soil to be investigated; and

FIG. 4 is an illustration showing a close up detail of a centralizer and guide block for the tube and probe of the instrument according to an embodiment of the present invention.

### DETAILED DESCRIPTION OF THE INVENTION

Whenever in the figures the same reference numerals are applied, these numerals refer to the same parts.

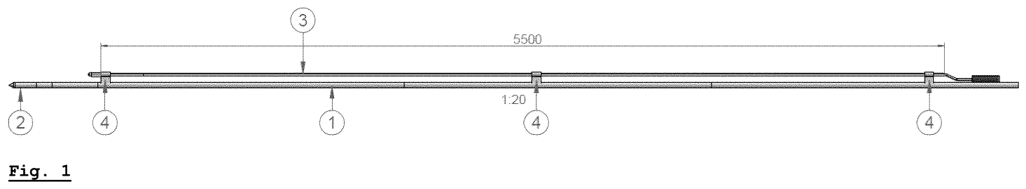

Referring to FIG. 1, a cone penetrometer test (CPT) tube 1 is shown with a forward pointing cone 2 equipped to be pushed into a soil at a controlled rate, wherein at a fixed distance outside and adjacent to the tube 1 a longitudinally extending measurement probe 3 is provided which is equipped for measuring thermal properties of the soil in which the tube 1 is to be pushed. It is clear from FIG. 1 that the longitudinally extending measurement probe 3 extends along more than 90% of the tube 1.

FIG. 1 further shows that the longitudinally extending measurement probe 3 is fixed to the tube 1 with two or more outriggers 4 so as to ensure that the measurement probe 3 can be pushed into the soil together with the tube 1 while the tube 1 and the probe 3 maintain their relative position with reference to each other.

The longitudinally extending measurement probe 3 is provided with a heat supply portion and/or a cooling portion. The way this can be implemented is known to the skilled person and is therefore not shown. Examples are that the heat supply portion and the cooling portion can be either passive or active. In the passive mode friction between tube 1 and probe 3 and the soil can be used for heating. This can also be done active with dedicated heating elements. Active cooling is of course also possible.

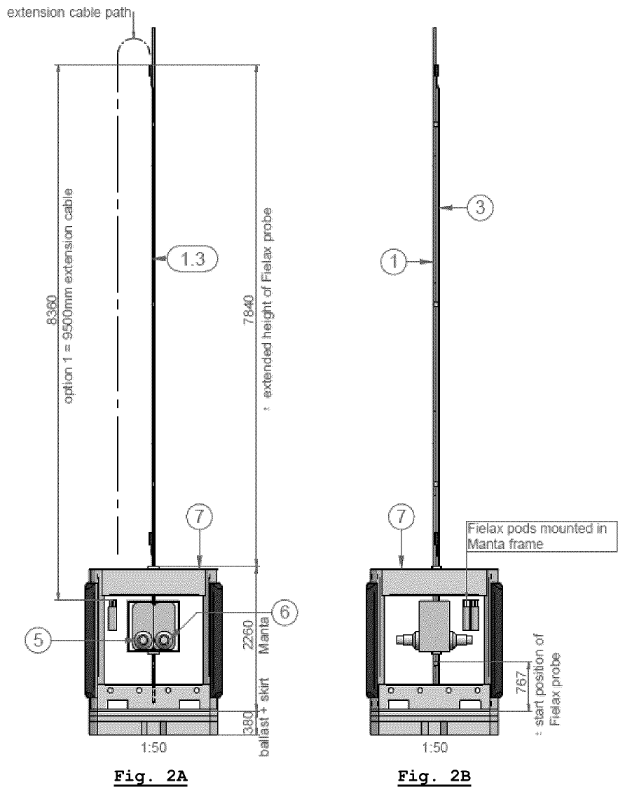

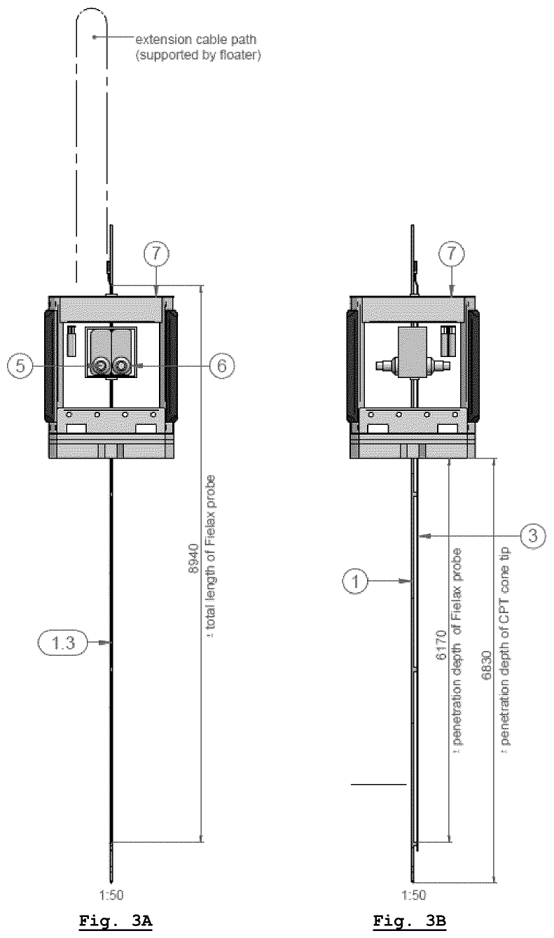

FIGS. 2A and 2B show the instrument of the invention in two different side views prior to entering into a soil to be investigated, whereas FIGS. 3A and 3B show the instrument of the invention in two different side views after entering into the soil to be investigated. To enable pushing in or pulling out of the combined tube 1 and probe 3, the tube 1 and probe 3 are mounted in driving frame 7. The figures show hydraulic engines 5, 6 that are arranged to drive a driving chain with clamping blocks to drive tube 1 and probe 3. Other driving options are of course feasible also, such as a piston drive, hydraulic rams etc.

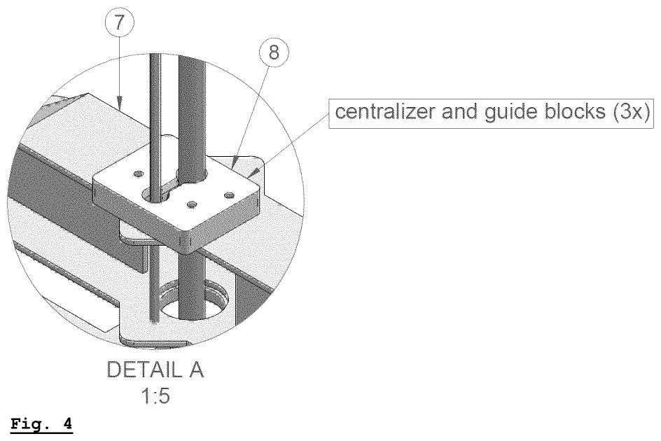

FIG. 4 is a detail view showing that frame 7 is further provided with at least one centralizer and guide block 8 for tube 1 and probe 3.

Note that in the specification and claims, “about” or “approximately” means within twenty percent (20%) of the numerical amount cited.

Although the invention has been discussed in the foregoing with reference to an exemplary embodiment of the instrument of the invention, the invention is not restricted to this particular embodiment which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Embodiments of the present invention can include every combination of features that are disclosed herein independently from each other. Although the invention has been described in detail with particular reference to the disclosed embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another. The terms, “a”, “an”, “the”, and “said” mean “one or more” unless context explicitly dictates otherwise.

What is claimed is:

1. A cone penetrometer test (CPT) tube comprising:
a forward pointing cone equipped to be pushed into a soil at a controlled rate; and
a longitudinally extending measurement probe disposed at a fixed distance outside and adjacent to the tube and equipped for measuring thermal properties of the soil in which the tube is pushed, wherein the longitudinally extending measurement probe is fixed to the tube with two or more outriggers so as to ensure that the measurement probe can be pushed into the soil together with the tube while the tube and the probe maintain their relative position with reference to each other, and wherein the longitudinally extending measurement probe comprises a heat supply portion and/or a cooling portion.

2. The cone penetrometer test (CPT) tube of claim 1, wherein the longitudinally extending measurement probe extends along at least about 1 meter of the tube.

3. The cone penetrometer test (CPT) tube of claim 1, wherein the tube and probe are mounted in a driving frame comprising at least one centralizer and guide block for the tube and probe.

4. The cone penetrometer test (CPT) tube of claim 3, wherein the driving frame comprises a set of driving rolls.
